(19) United States
(12) Patent Application Publication
(10) Pub. No.: US 2009/0084698 A1
(43) Pub. Date: Apr. 2, 2009

(54) Absorbent Product for Protection from Ultraviolet Ray

(76) Inventors: Kazuharu Ito, Nishinomiya (JP); Yasuko Shiomichi, Kobe (JP); Dennis San Agustin Casalme, Kobe (JP)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/211,403

(22) Filed: Sep. 16, 2008

Related U.S. Application Data
(60) Provisional application No. 60/995,641, filed on Sep. 27, 2007.

(51) Int. Cl. A61B 19/02; A61F 13/58

(52) U.S. Cl. 206/438; 604/385.03

(57) ABSTRACT

An absorbent product comprises (i) a package and (ii) at least one disposable absorbent article contained in the package. The package comprises a window area and an ultraviolet barrier area. The window area allows more light transmittance than the ultraviolet barrier area. The ultraviolet barrier area allows less ultraviolet transmittance than the window area. The absorbent article comprises a liquid permeable topsheet, a liquid impermeable backsheet, an absorbent core disposed between the topsheet and the backsheet, and a reinforcement adhesive having a basis weight of not less than about 10 g/m². The absorbent article comprises a graphic which is at least partially visible through the window area. The reinforcement adhesive is covered by the ultraviolet barrier area.

### CROSS REFERENCE TO RELATED APPLICATION

This application claims the benefit of U.S. Provisional Application No. 60/995641, filed Sep. 27, 2007.

### FIELD OF THE INVENTION

The present invention relates to an absorbent product comprising a package and at least one absorbent article contained in the package.

### BACKGROUND OF THE INVENTION

Disposable absorbent articles are devices which are typically worn in the crotch region of a wearer. The absorbent articles include diapers, training pants, sanitary napkins, pantiliners, incontinent pads, sweat-absorbent underarm pads, nursing pads, human waste management devices and the like. Among them, diapers and training pants, for example, are worn to contain discharged materials such as urines and feces and to isolate these materials from the body of the wearer and from the wearer's surroundings. A wide variety of disposable absorbent articles are currently used for the collection of discharged materials.

Recently, it has been recognized that an absorbent product comprising a package having a transparent or a translucent window is preferred by consumers since such a window can actually show the absorbent articles contained in the package and thus consumers can directly get the information about the absorbent articles through the window. This may be important for recent disposable absorbent articles which have aesthetic features (e.g., a printed graphic thereon) to draw consumers' attention.

It is known in the art that diapers are often provided with a graphic on the backsheet wherein the graphic is visible through a window of a package. Such a package is convenient to attract the consumers not only at the markets but also at home.

It has been found that providing a transparent or a translucent window in a package may cause serious problems to the absorbent articles contained therein. For example, diapers in a package which has a transparent window may be affected by the ultraviolet ray which comes through the window. Because a transparent or translucent window tends to allow ultraviolet transmittance, a part of the absorbent article which faces the window may be deteriorated by the ultraviolet ray.

Generally, a diaper contains an adhesive to join various elements of the diaper. The adhesive tends to be vulnerable to the ultraviolet ray. The adhesive may be discolored or stiffened when it receives the ultraviolet ray. Discoloration (or yellowing) is perceivable when the diaper is visible through the window of the package. Discoloration is also perceivable after the diaper is taken out of the package. Stiffening is perceivable when the user actually treats the diaper and stiffening may cause the diaper to increase discomfort when it is worn. When a large amount of an adhesive is used, the discoloration of the adhesive may be noticeably recognized. Like this, the influence of the ultraviolet ray on the adhesive contained in the diaper may be easily perceivable. Furthermore, some retailers build merchandizing shelves outside the stores where a sun-shade is not available. When the package having a window is exposed to sunlight, and the adhesive in the diaper contained in the package receives the ultraviolet ray through the window, the adhesive may be deteriorated. Consequently, the appearance and the quality of the diaper may be damaged.

Thus, there is a need for a package which shows a graphic of the diaper in order to appeal to the consumers, and at the same time prevents deterioration of the adhesive contained in the diaper.

### SUMMARY OF THE INVENTION

The present invention relates to an absorbent product comprising (i) a package and (ii) at least one disposable absorbent article contained in the package. The package comprises a window area and an ultraviolet barrier area. The window area allows more light transmittance than the ultraviolet barrier area. The ultraviolet barrier area allows less ultraviolet transmittance than the window area. The absorbent article extends in a longitudinal direction and in a transverse direction orthogonal to the longitudinal direction. The absorbent article comprises a liquid permeable topsheet, a liquid impermeable backsheet, an absorbent core disposed between the topsheet and the backsheet, and a reinforcement adhesive having a basis weight of not less than about 10 g/m². The absorbent article comprises a graphic which is at least partially visible through the window area. The reinforcement adhesive is covered by the ultraviolet barrier area.

### BRIEF DESCRIPTION OF THE DRAWINGS

While the specification concludes with claims which particularly point out and distinctly claim the invention, it is believed that the present invention will be better understood from the following description of preferred embodiments taken in conjunction with the accompanying drawings, in which like reference numerals identify identical elements and wherein:

FIG. 1 illustrates a perspective front view of a disposable absorbent article comprising a graphic;

FIG. 2 illustrates an expansion view of the disposable absorbent article of FIG. 1 whose side seams are detached;

FIG. 3 illustrates a cross sectional view of the disposable absorbent article of FIG. 2 taken along by 3-3′ line;

FIG. 4 illustrates a side view of a package comprising a window area through which a graphic is visible;

FIG. 5 illustrates a perspective view of a package containing a plurality of disposable absorbent articles inside;

FIG. 6 illustrates an embodiment of a side view of an absorbent product showing the relationship between the disposable absorbent article and the package;

FIG. 7 illustrates a perspective rear view of a disposable absorbent article comprising a graphic and a disposable tape on the external surface of the nonwoven over the backsheet;

FIG. 8 illustrates a cross sectional view of the disposable absorbent article of FIG. 7 taken along by 8-8′ line;

FIG. 9 illustrates a side view of a package showing the relationship between the disposable absorbent article and the package; and

FIG. 10 illustrates a cross sectional view of the disposable absorbent article of FIG. 2 taken along by 10-10′ line.

### DETAILED DESCRIPTION OF THE INVENTION

Herein, “comprise” and “include” mean that other elements and/or other steps which do not affect the end result can be added. Each of these terms encompasses the terms “consisting of” and “consisting essentially of”.

Herein, “absorbent article” refers to articles which absorb and contain discharged materials, and is intended to include diapers, training pants, sanitary napkins, pantiliners, incontinence pads, sweat-absorbent underarm pads, nursing pads, adult incontinence diapers, human waste management devices and the like.

Herein, “disposable” refers to articles which are intended to be discarded after a single use, composted, or otherwise disposed of in an environmentally compatible manner.

Herein, “diaper” refers to a garment generally worn by toddlers and incontinent persons that is worn about the lower torso of the wearer which is intended to absorb and contain discharged materials from the body (e.g., urine, feces). Diapers include, for example, pull-on diapers and taped diapers. Pull-on diapers refer to diapers which have a defined waist opening and a pair of leg openings and which are pulled onto the body of the wearer by inserting the legs into the leg openings and pulling the article up over the waist. Taped diapers refer to diapers generally include a front and a rear waist section releasably and/or refastenably connected by a fastening system.

Herein, “joined” encompasses configurations in which an element is directly secured to another element by affixing the element directly to the other element; configurations in which the element is indirectly secured to the other element by affixing the element to intermediate member(s) which in turn are affixed to the other element; and configurations in which one element is integral with another element, i.e., one element is essentially part of the other element.

Herein, “body facing surface” refers to surfaces of absorbent articles and/or their component members which face the body of the wearer. “External surface” refers to the opposite surfaces of the absorbent articles and/or their component members that face away from the wearer when the absorbent articles are worn.

Absorbent articles and components thereof, including a liquid permeable topsheet, a liquid impermeable backsheet and an absorbent core disposed between the topsheet and the backsheet, and any individual layers of their component members, have a body facing surface and an external surface.

In the present invention, “absorbent product” comprises a package and at least one disposable absorbent article contained in the package.

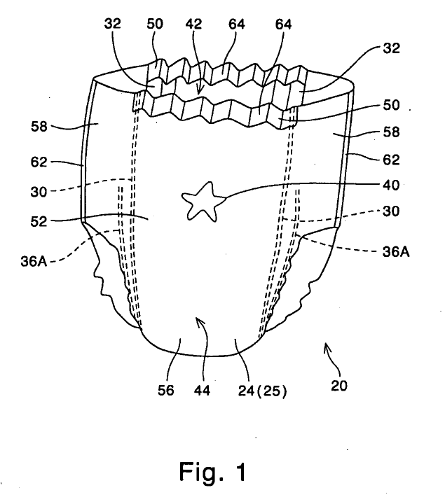

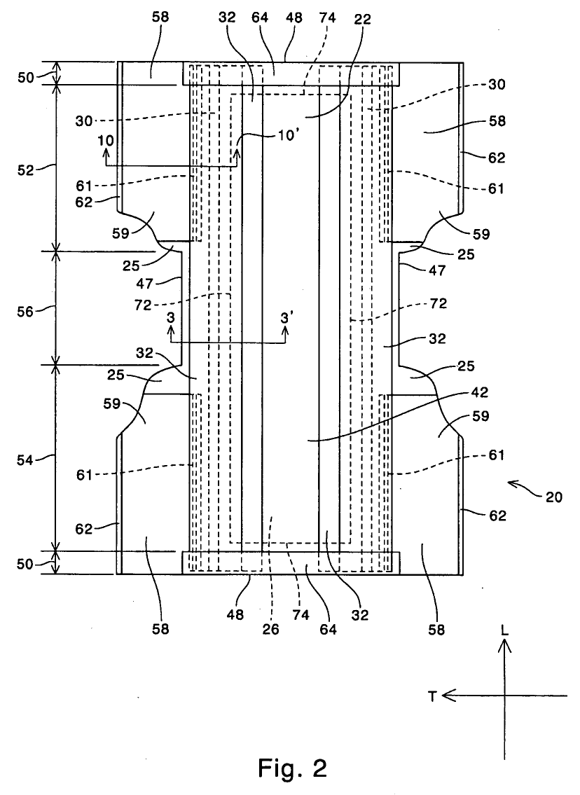

FIGS. 1 and 2 illustrate a disposable pull-on diaper which is representative of a disposable absorbent article to be contained in a package. The diaper 20 comprises a liquid permeable topsheet 22, a liquid impermeable backsheet 24 attached to the topsheet 22 and an absorbent core 26 disposed between the topsheet 22 and the backsheet 24. The diaper 20 has a body facing surface 42 and an external surface 44 opposed to the body facing surface 42. The diaper 20 comprises a leg cuff 32 disposed on the body facing surface 42 of the diaper 20.

The diaper 20 extends in a longitudinal direction L and in a transverse direction T. “Longitudinal” refers to a line, axis or direction in the plane of the diaper 20 that is generally aligned with (e.g., approximately parallel to) a vertical plane which bisects a standing wearer into left and right body halves when the diaper 20 is worn. “Transverse” or “lateral”, are interchangeable, and refer to a line, axis or direction which lies within the plane of the diaper 20 that is generally perpendicular to the longitudinal direction L. The diaper 20 comprises a pair of longitudinal side edges 47 and a pair of transverse end edges 48. The diaper 20 comprises a pair of waist regions 50, a front region 52, a rear region 54, a crotch region 56 and a pair of elastic side regions 58 which connect the front region 52 and the rear region 54. Each of the elastic side regions 58 is formed from two separate elastic portions and they are joined together to form side seams 62.

The topsheet 22 may be compliant, soft feeling, and non-irritating to the wearer's skin. The topsheet 22 is liquid permeable or pervious, permitting discharged materials to readily penetrate through its thickness. A liquid permeable topsheet 22 may be manufactured from a wide range of materials such as woven and nonwoven materials (e.g., a nonwoven web of fibers); polymeric materials such as apertured formed thermoplastic films, apertured plastic films, and hydroformed thermoplastic films; porous foams; reticulated foams; reticulated thermoplastic films; and thermoplastic scrims. When the topsheet 22 includes a nonwoven web, the web may be manufactured by a wide number of known techniques. For example, the web may be spunbonded, carded, wet-laid, melt-blown, hydroentangled, combinations of the above, or the like. The body facing surface of the topsheet 22 can be made hydrophilic by the treatment with a surfactant. The external surface of the topsheet 22 may be attached to the absorbent core 26.

The backsheet 24 may be impervious to discharged materials and may be manufactured from a thin plastic film, and other flexible liquid impervious materials may be also used. The backsheet 24 prevents discharged materials absorbed and contained in the absorbent core 26 from leaking out of the absorbent article 20. The backsheet 24 may thus include woven or nonwoven materials, polymeric films such as thermoplastic films of polyethylene or polypropylene, or composite materials such as a film-coated nonwoven material. The backsheet 24 may include a single layer material, or plurality of layer materials. In one embodiment, the backsheet 24 may be a single layer polyethylene film. The backsheet 24 may have a microporous structure which can permit vapors to escape from the absorbent core (called “breathable backsheet”) while still preventing discharged materials from passing through the backsheet 24.

The backsheet 24 may be further covered by nonwoven 25. In the embodiment of FIG. 1, the nonwoven 25 covers the external surface of the backsheet 24. The nonwoven 25 may make the diaper 20 feel soft. The nonwoven 25 generally has a loose texture, and an object looks blurred when it is covered by the nonwoven 25.

The absorbent core 26 is capable of receiving, absorbing and/or retaining discharged materials from the body. The absorbent core 26 may be compressible, conformable and non-irritating to the wearer's skin. The absorbent core 26 has a pair of longitudinal side edges 72 and a pair of transverse end edges 74. The absorbent core 26 may be formed by a single layer material or a plurality of layer materials. The absorbent core 26 may include any of a wide variety of liquid-absorbent materials known in the art, such as comminuted wood pulp, which is generally referred to as airfelt. The absorbent core 26 may comprise a multi-bonded air laid nonwoven material. The absorbent core 26 may be manufactured in a wide variety of sizes and shapes. The absorbent core 26 thus can take any shape in its top plan view. Shapes for the absorbent core 26 may include an oval, a rectangle, an hourglass, a circle, a square, and any other shape. In the embodiment of FIG. 2, the core 26 takes a rectangle shape.

Referring to FIGS. 1 and 2, the diaper 20 comprises a pair of elastic waist bands 64 in the waist regions 50. The elastic waist bands 64 are disposed longitudinally outside the transverse ends 74 of the absorbent core 26. The elastic waist bands 64 provide the waist regions 50 of the diaper 20 with elasticity.

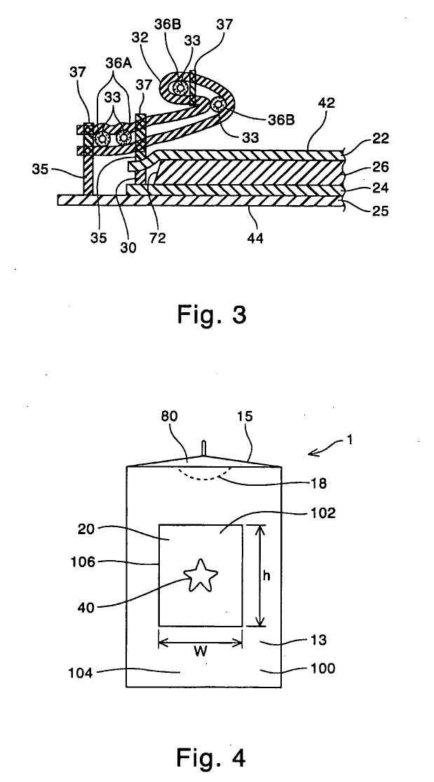

Referring to FIGS. 1, 2 and 3, the diaper 20 further comprises a pair of leg cuffs 32 for providing improved containment of discharged materials. Each leg cuff 32 may comprise several different embodiments for blocking or reducing the leakage of discharged materials in the leg regions on the body facing surface 42 of the diaper 20. The leg cuff 32 extends in a substantially longitudinal direction L to overlap the elastic waist bands 64 in order to block or reduce leakage of discharged materials from the diaper 20. In the embodiment of FIG. 3, the leg cuff 32 is secured to the topsheet 22 by an attachment means 35. Additionally or alternatively, the leg cuff 32 may be secured to the backsheet 24 and/or the nonwoven 25 by an attachment means 35. The attachment means 35 includes, for example, an adhesive, a thermal bonding, and the like. The material for the leg cuff 32 may be repellent nonwoven. The leg cuff 32 may be breathable and liquid impervious. The leg cuff 32 comprises elastics 33 which provide the leg cuff 32 with elasticity. The elastics 33 are at least partially coated with elastic adhesives 36. The leg cuff 32 may comprise a plurality of continuous bonds 37 to fix and maintain the folded structure of the leg cuff 32. The continuous bonds 37 may include, for example, an adhesive, a thermal bonding, and the like. In the embodiment of FIG. 3, the continuous bond is a thermal bonding. The continuous bonds 37 may extend in a substantially longitudinal direction L between a pair of transverse end edges 48 of the diaper 20.

The diaper 20 comprises a graphic 40 which is visible from outside. The graphic 40 may be disposed in the waist region 50, the front region 52, the rear region 54, and/or the side region 58 of the diaper 20. In order to draw consumers' attention, the graphic 40 may be disposed in the front region 52 and the rear region 54. The graphic 40 may extend from the front region 52 to the rear region 54 as a continuous graphic. Referring to FIG. 1, the diaper 20 comprises a graphic 40 printed on the external surface of the backsheet 24. The term “graphic” may refer, but is not limited, to an image, a design, a pattern, symbology, indicia, or the like. “Graphic” includes, for example, animals (e.g., dogs, cats, bears, squirrels, tigers, lions, mice, foxes, and the like); birds (e.g., swallows, sparrows, hawks, ducks, eagles, swans, and the like); human beings; plants such as flowers (e.g., dandelions, roses, tulips, cherry blossoms, sunflowers, carnations, and the like), trees and leaves; stars; moons; cartoon characters; toys (e.g., dolls, bats, balls, rackets, and the like); electric instruments (e.g., mobile phones, computers, and the like); ornaments (e.g., frills, ribbons, buttons, belts, neckties, caps, hats, and the like); garment-like patterns (e.g., denims, borders, stripes, checks, polka dots, and the like); seasonal things or goods such as a snowman; landscapes; and the like. The graphic 40 may be printed on a part of the backsheet 24 or on the substantially whole surface of the backsheet 24. The graphic 40 may be printed on the body facing surface of the backsheet 24. In the embodiment of FIG. 1, the graphic 40 is printed on the backsheet film and the backsheet film is further covered by the nonwoven 25. Therefore, the graphic 40 looks blurred when it is covered by the nonwoven 25 and the diaper 20 comprising such a graphic 40 looks noble and refined. The graphic 40 may be printed on the nonwoven 25. The graphic 40 may be printed on another material than the backsheet 24 or the nonwoven 25 (e.g. a polymeric film, a sheet of paper, and the like) and the printed material may be joined on the backsheet 24 or the nonwoven 25. The graphic 40 can be printed by any conventional printing methods or technologies known in the art, including, but not limited to, a gravure printing, a flexo printing, an offset printing, an ink jet printing, and the like. The graphic 40 may be positioned in the waist region 50, the front region 52, the rear region 54, the crotch region 56 and/or the side region 58 of the diaper 20.

The absorbent article 20 comprises an adhesive. “Adhesive” refers to a material which is contained in the absorbent articles 20 and secures one portion to another. Adhesives comprise, for example, construction adhesives and reinforcement adhesives. “Construction adhesives” may be used to construct the absorbent article 20 by securing one portion to another mainly for the purpose of adhesion. The construction adhesives comprise, for example, adhesives which secure the nonwoven 25 to the backsheet 24, adhesives which secure the backsheet 24 to the absorbent core 26, adhesives which secure the absorbent core 26 to the topsheet 22, adhesives which secure the elastics of the waist band 64 to the waist region 50, and the like. An adhesive having a basis weight of not more than about 10 g/m² may be used for the construction adhesives. An adhesive having a basis weight of more than about 10 g/m², if used for the construction adhesives, may lead to wasting the amount of an adhesive. “Reinforcement adhesives” may be used to locally reinforce the function of the absorbent article 20 in addition to the purpose of adhesion. In order to reinforce the function of the absorbent article 20, an adhesive having a basis weight of not less than about 10 g/m² may be used. An adhesive having a basis weight of less than about 10 g/m² may not be enough to ensure the function. The basis weight of the adhesive may be not more than about 200 g/m². An adhesive having a basis weight of more than about 200 g/m² may make the diaper 20 severely stiffened. The reinforcement adhesives comprise, for example, leakage prevention adhesives, disposable tape adhesives, side region attachment adhesives, elastic adhesives, and the like. As the basis weight of the reinforcement adhesive is not less than about 10 g/m², the deterioration by the ultraviolet ray (e.g., discoloration) may be perceivable from outside.

Leakage prevention adhesives are used to block or reduce the leakage of discharged materials from the absorbent core 26. The leakage prevention adhesive 30 may be applied on the body facing surface of the backsheet 24 in a substantially longitudinal direction L and disposed transversely outside the longitudinal side edges 72 of the absorbent core 26. The leakage prevention adhesive 30 may be applied substantially continuously between a pair of transverse end edges 48 of the diaper 20. In the embodiment of FIGS. 2 and 3, the diaper 20 comprises a leakage prevention adhesive 30 which secures the backsheet 24 to the topsheet 22 to block or reduce the leakage through between the topsheet 22 and the backsheet 24. In another embodiment, the leakage prevention adhesive may secure the backsheet 24 to the leg cuff 32 to block or reduce the leakage through between the backsheet 24 and the leg cuff 32. The leakage prevention adhesive 30 may have a basis weight of not less than about 20 g/m² to prevent the leakage of discharged materials. The basis weight of the leakage prevention adhesive 30 can be not less than about 30 g/m². The basis weight of the leakage prevention adhesive 30 may be not more than about 150 g/m².

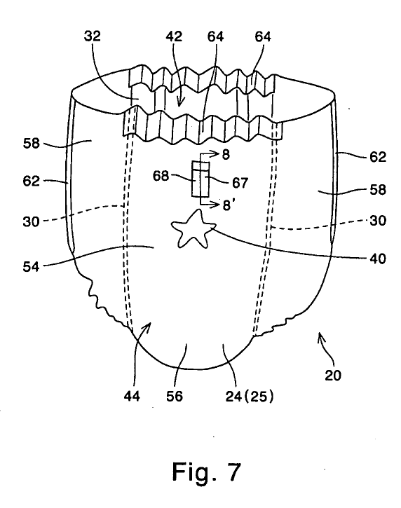

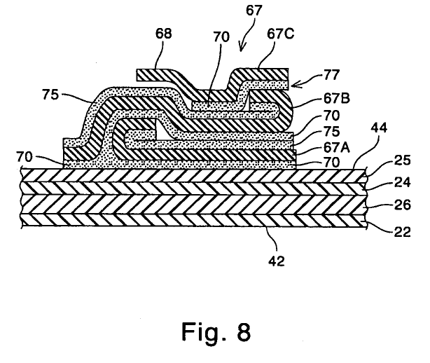

Disposable tape adhesives are used for a disposable tape. Referring to FIG. 7, the absorbent article 20 comprises a disposable tape 67 secured to the external surface 44 of the rear region 54 of the diaper 20. FIG. 8 illustrates a partial cross sectional view of the absorbent article of FIG. 7 cut off by 8-8′ line. The sectional view illustrates the structure of the disposable tape 67. The disposable tape 67 may be used to secure a soiled diaper containing discharged materials for disposal. The disposable tape 67 may be secured to the front region 52 or the rear region 54 of the absorbent article 20. In the embodiment of FIG. 7, the pull-on diaper 20 comprises a disposable tape 67 secured to the external surface 44 of the rear region 54 of the diaper 20. The disposable tape 67 is used to fasten the used diaper containing the discharged materials and thus soiled diaper is folded down to wrap the discharged materials. The disposable tape 67 comprises a disposable tape adhesive 70.

Referring to FIG. 8, the disposable tape 67 comprises a first section 67A, a second section 67B and a third section 67C. The first section 67A is secured by a disposable tape adhesive 70 to the nonwoven 25, and is secured by a disposable tape adhesive 70 to the second section 67B at a first adhesion portion 76. The second section 67B is secured by a disposable tape adhesive 70 to the third section 67C at a second adhesion portion 77. The first section 67A may have a release agent 75 on the surface facing the second section 67B. The second section 67B may also have a release agent 75 on the surface facing the third section 67C. A suitable release agent 75 can be a coating of silicon or a release liner made of polyethylene. Overall, the three sections of the disposable tape 67A, 67B and 67C compose a Z-folding on the external surface of the nonwoven 25.

The third section 67C comprises a disposable tape tab 68 on the opposite end to the second adhesion portion 77. The user can pull the disposable tape tab 68 to unfold the disposable tape 67 for disposal. When the disposable tape tab 68 is pulled, the first section 67A is partially detached from the second section 67B. Similarly, the second section 67B is partially detached from the third section 67C. Nevertheless, the three sections 67A, 67B and 67C are partially secured to each other with the disposable tape adhesive 70 at the first adhesion portion 76 and at the second adhesion portion 77. Therefore when the disposable tape tab 68 is pulled, the three sections 67A, 67B and 67C compose a long disposable tape which is used for rolling a soiled diaper. The soiled diaper is rolled up to conceal the discharged materials inside and the rolled up configuration of the diaper is fixed with the disposable tape 67. A various kinds of materials may be used for the disposable tape 67. The material used for the disposable tape 67 illustrated in FIGS. 7 and 8 is a polypropylene film.

The disposable tape adhesive 70 may be used to secure the disposable tape 67 to the diaper 20. The disposable tape adhesive 70 may also be used to attach two portions of the disposable tape 67 such as the first section 67A and the second section 67B, or the second section 67B and the third section 67C. The disposable tape 67 may be secured to the nonwoven 25 not to be easily detached when the diaper 20 is worn. Also the first section 67A is secured to the second section 67B, and the second section 67B is secured to the third section 67C not to be easily detached when the diaper 20 is worn. The adhesibility of the disposable tape adhesive 70 may be strong enough to keep the disposable tape 67 secured to the nonwoven 25 when the disposable tape tab 68 is pulled. The nonwoven 25 has a relatively rough surface and therefore the disposable tape adhesive 70 may have a basis weight of not less than about 20 g/m² to secure the disposable tape 67 to the nonwoven 25 firmly. The adhesibility of the disposable tape adhesive 70 may be strong enough to keep the first section 67A secured to the second section 67B at the first adhesion portion 76 and to keep the second section 67B secured to the third section 67C at the second adhesion portion 77 when the disposable tape tab 68 is pulled. The basis weight of the disposable tape adhesive 70 can be not less than about 25 g/m². The basis weight of the disposable tape adhesive 70 may be not more than about 150 g/m². Materials used for the disposable tape 67 may include, but are not limited to, polymeric film, such as polypropylene films, polyethylene films, co-extruded polyethylene and ethylene vinyl acetate films and the like. The material for the disposable tape may be of biodegradable, recyclable, non-biodegradable or non-recyclable materials. In the embodiment of FIGS. 7 and 8, the disposable tape is polypropylene.

Side region attachment adhesives are used to secure an elastic portion of the side region to the leg cuff 32. In the embodiment of FIG. 10, an elastic portion 59 comprises an elastic film 60 whose body facing surface and external surface are covered by nonwoven 25 and 63 respectively to provide softness. The elastic portion 59 may have elasticity. Referring to FIG. 2, the side region attachment adhesive 61 extends in a substantially longitudinal direction L. The side region attachment adhesive 61 is disposed transversely outside the leakage prevention adhesive 30. When a caregiver lets the wearer put on the diaper 20, the caregiver may stretch the diaper 20 to ensure easy wearing. Then the side region attachment adhesive 61 may receive a strong stretch force. In order to resist to the stretch force, the side region attachment adhesive 61 may have a basis weight of not less than about 40 g/m². The basis weight of the side region attachment adhesive 61 can be not less than about 50 g/m². The basis weight of the side region attachment adhesive 61 may be not more than about 180 g/m².

Leg elastic adhesives are used to fix the position of a leg elastic 33 inside the leg cuff 32. The leg elastic 33 is used to fit the diaper 20 to the wearer's body. In the embodiment of FIG. 3, the leg elastic 33 may be at least partially coated by the leg elastic adhesive 36. The leg elastic adhesive 36 may secure the leg elastic 33 to the leg cuff 32. The leg elastic 33 extends in a substantially longitudinal direction L along the leg cuff 32. In the embodiment of FIGS. 1 and 3, a leg elastic adhesive 36A is disposed transversely outside of the leakage prevention adhesive 30. In the embodiment of FIG. 3, a leg elastic adhesive 36B is located transversely inside the leakage prevention adhesive 30. In order to fix the leg elastic 33 firmly, the basis weight of the leg elastic adhesive 36 may be not less than about 15 g/m². The basis weight of the leg elastic adhesive 36 can be not less than about 20 g/m². The basis weight of the leg elastic adhesive 36 may be not more than about 160 g/m².

Adhesives may be selected from pressure sensitive adhesives, non-pressure sensitive adhesives, hot melt adhesives, and the like. Any adhesive materials known in the art can be used for the adhesive such as SIS (styrene-isoprene-styrene block copolymer), SBS (styrene-butadiene-styrene block copolymer), polyolefin, and the like. For example, the following adhesives may be used; Findley H2401 (SIS Polymer), Findley H2861 (SIS Polymer), Fuller 3166 (Polyolefin), and NSC 519 (SBS Polymer).

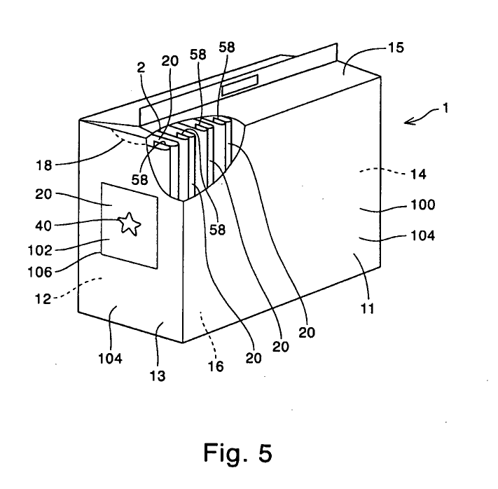

FIG. 4 illustrates a side view of an absorbent product comprising a package and a disposable absorbent article therein. FIG. 5 illustrates a perspective view of the package of FIG. 4. The package 100 may be any shape known in the art. For example, the package 100 may have a polyhedral shape defining or forming a polyhedral enclosure. The package 100 is a shape of a parallelepiped including a front panel 11, a rear panel 12 opposed to the front panel 11, a pair of side panels 13 and 14 which connect the front panel 11 and the rear panel 12, a top panel 15 which connects the front panel 11, the rear panel 12 and the side panels 13 and 14, and a bottom panel 16 opposed to the top panel 15.

The package 100 containing at least one absorbent article 20 may include those constructed as cartons and/or flexible packages, such as pouches and bags. The package 100 may be formed by any suitable material and can take any structure known in the art. Materials used to construct packages may include, but are not limited to, polymeric film, such as polypropylene films, polyethylene films, co-extruded polyethylene and ethylene vinyl acetate films and the like, paperboard and coated paper. The package material may be of biodegradable, recyclable, non-biodegradable or non-recyclable materials. In the embodiment of FIGS. 4 and 5, the package 100 is a flexible bag which is formed by a thin film material. The package 100 may be formed through manipulation of a single sheet of material, such as folding, by attaching multiple sheets to one another or a combination thereof. The package 100 may be sealed or adhered by means known in the art, such as heat seal, ultrasonics, adhesives, hook and loop fasteners and the like. A film material for the package 100 illustrated in FIGS. 4 and 5 is a polyethylene film.

An opening device for retrieving an absorbent article 20 may be provided at an arbitrary area of the package 100. In the embodiment of FIGS. 4 and 5, an opening device 18 for retrieving a diaper 20 is provided on the side panel 13. The opening device 18 may have an enough size (e.g., the length) so that the diapers 20 can be retrieved easily by the user. The opening device 18 can take any structure, shape and dimension known in the art. The opening device 18 may include a line of weakness which extends within the side panel 13. The line of weakness may include a line of perforation formed in the side panel 13. The line of weakness may extend into the other panels such as the top panel 15 and the bottom panel 16, from the side panel 13. The two side portions of the package 100 are closed by forming a top gusset structure 80 which is formed by sealing the material film.

The package 100 comprises a window area 102 and an ultraviolet barrier area 104. The “window area” refers to an area of the surface of the package 100 which allows light transmittance so that the graphic 40 of the diaper 20 contained in the package 100 may be at least partially visible therethrough. The “ultraviolet barrier area” refers to an area of the surface of the package 100 which blocks or reduces the ultraviolet transmittance. The window area 102 allows more light transmittance than the ultraviolet barrier area 104. The ultraviolet barrier area 104 allows less ultraviolet transmittance than the window area 102.

The window area 102 may be transparent or translucent to allow the graphic 40 to be visible therethrough. In the embodiment of FIG. 4, the window area 102 is transparent and the ultraviolet barrier area 104 is opaque. In another embodiment, the window area 102 may be translucent and the ultraviolet barrier area 104 may be opaque. In still another embodiment, the window area 102 may be transparent and the ultraviolet barrier area 104 may be translucent. The light transmittance and the ultraviolet transmittance are distinguished between these two areas. Otherwise, both the window area 102 and the ultraviolet barrier area 104 may be translucent and these two areas are distinguished by differentiating the light transmittance and ultraviolet transmittance from each other. The ultraviolet barrier area 104 may be distinguished from the window area 102 by the color difference. For example, the ultraviolet barrier area 104 may have a strong color which blocks or reduces light transmittance and ultraviolet transmittance and the window area 102 may have a light color which allows more light transmittance and ultraviolet transmittance than the ultraviolet barrier area 104.

The window area 102 and the ultraviolet barrier area 104 can be formed by any means known in the art. A window area 102 may be prepared by using a transparent or translucent material known in the art (e.g. polymeric films such as thermoplastic films of polyethylene or polypropylene, or a composite material such as a film-coated nonwoven material) as it is or by coloring the material. An ultraviolet barrier area 104 may be prepared by coloring the transparent or translucent material to have less light transmittance and less ultraviolet transmittance. The transparent or translucent material may be partially colored, for example by printing. In one embodiment, the transparent or translucent material may be partially printed and the rest of the material is left unprinted. The printed area may be an ultraviolet barrier area 104 and the unprinted area may be a window area 102. In another embodiment, substantially all the area of the transparent or translucent material film may be printed to provide a translucent window area and an opaque area. In such a case, the transparent or translucent material film may be partially printed to be at least translucent to correspond to the window area 102 and the rest of the material film may be printed to be opaque to correspond to the ultraviolet barrier area 104. The material film can be printed by any conventional printing methods or technologies known in the art, including, but not limited to, a gravure printing, a flexo printing, an offset printing, an ink jet printing, and the like.

The window area 102 may be disposed on any place of the package 100 as far as the window area 102 allows the graphic 40 to be visible. For example, the window area 102 may be disposed on the front panel 11, the rear panel 12, the side panel 13 or 14, the top panel 15, the bottom panel 16 or a combination thereof. The window area 102 may be disposed to extend more than one panel. A plurality of window areas 102 may be disposed on the package 100. In the embodiment of FIGS. 4 and 5, the graphic 40 is disposed on the diaper 20 and the graphic 40 may face the window area 102. The retailers may place the absorbent product 1 to show the window area 102 in the side panel 13 so that the graphic 40 may be perceivable from outside. The graphic 40 which is perceivable through the window area 102 may serve to attract the customers.

The window area 102 can take any shape such as a circle, a square, a rectangle, a trapezoid, an ellipse, a triangle, an oval, a semi-circle, a sector, a star or any other shape. The shape of the window area 102 illustrated in FIGS. 4 and 5 is a rectangle. The window area 102 can have any sizes or dimensions (e.g., the length and the width for a rectangular window, the diameter for a circle window) as far as the graphic 40 can be effectively visible through the window area 102. “Effectively” refers to the degree of the visibility for the consumer to identify the graphic 40 on the absorbent article 20 from outside. The window area 102 may be smaller than the panel comprising the window area 102. In the embodiment of FIGS. 4 and 5, the size of the window area 102 is smaller than that of the side panel 13. For example, the width W of the window area 102 may be between about 40 mm and about 100 mm and the height H of the window area 102 may be between about 40 mm and about 200 mm. In the embodiment of FIGS. 4 and 5, the window area 102 may occupy between about 5% and about 80% of the total area of the side panel 13 comprising the window area 102. The window area 102 enables the consumer to understand directly at the point of purchase that the diaper 20 is provided with a graphic 40. In order to show the graphic 40, the window area 102 is located to cover the center region 112 and the ultraviolet barrier area 104 is located to cover the side regions 114.

The ultraviolet barrier area 104 may be additionally or alternatively protected by an ultraviolet absorbing agent. Examples of commercially available ultraviolet absorbing agents include, for example, benzophenone type (Viosorb 110, Viosorb 130, EVERSORB 10, EVERSORB 11, EVERSORB 12), benzotriazole type (Viosorb 520, Viosorb 550, Viosorb 582, Viosorb 583, Viosorb 590, Viosorb 591, EVERSORB 70, EVERSORB 71, EVERSORB 72, EVERSORB 73, EVERSORB 74, EVERSORB 75, EVERSORB 76, EVERSORB 234, EVERSORB 78, EVERSORB 80, EVERSORB 81, Tinuvin 213, Tinuvin 234, Adekastab LA-31), hindered amine light stabilizer (HALS) (Sanol LS-770, Sanol LS-765, Tinuvin 622, Chimassorb 944, Cyassorb UV-3346, Adekastab LA-57, Chimassorb 119, EVERSORB 90, EVERSORB 91, EVERSORB 93, EVERSORB S02), benzoate type (Viosorb 80, SB-UVA 612) and the like.

The light transmittance ranges provide an effective view of the graphic 40 through the window area 102. The light transmittance of the window area 102 may be between about 15% and about 100%. Or the light transmittance of the window area 102 may be between about 25% and about 100%. The window area 102 may have more than about 20% greater light transmittance than the ultraviolet barrier area 104. A method for measuring the light transmittance will be described in the “Test Methods” section. The light transmittance of the ultraviolet barrier area 104 may be between about 0% and about 70% insofar as the window area 102 allows more light transmittance than the ultraviolet barrier area 104. Or the light transmittance of the ultraviolet barrier area 104 may be between about 0% and about 50%. Lower light transmittance can serve to provide lower ultraviolet transmittance.

The ultraviolet transmittance ranges may provide an effective protection of the reinforcement adhesive having a basis weight of not less than about 10 g/m² from the ultraviolet ray. The ultraviolet transmittance of the ultraviolet barrier area 104 may be between about 0% and about 50%. Or the ultraviolet transmittance of the ultraviolet barrier area 104 may be between about 0% and about 25%. The ultraviolet barrier area 104 may have less than about 20% lower ultraviolet transmittance than the window area 102. A method for measuring the ultraviolet transmittance will be described in the “Test Methods” section. The ultraviolet transmittance of the window area 102 may be between about 15% and about 100% insofar as the ultraviolet barrier area 104 allows less ultraviolet transmittance than the window area 102. Or the ultraviolet transmittance of the ultraviolet barrier area 104 may be between about 25% and about 100%. As is easily understood by a person skilled in the art, the lower ultraviolet transmittance means the higher ultraviolet blockade.

The absorbent product 1 comprises a package 100 and at least one absorbent article 20 in the package 100. The package 100 defines an interior space 2 for containing at least one absorbent article 20. The graphics 40 of the absorbent articles 20 may all be identical with one another or may be different from one another. In the embodiment of FIG. 5, a plurality of diapers 20 are compressed to form one stack within the interior space 2 of the package 100.

At least one diaper 20 is contained in the package 100 such that the graphic 40 may be visible through the window area 102. In the embodiment of FIGS. 4 and 5, a plurality of diapers 20 is stacked so that the front region 52 of one diaper 20 and the rear region 54 of another diaper may face each other. The diaper 20 is folded so that the side regions 58 are at least partially hidden between the front region 52 and the rear region 54. Accordingly, the side region attachment adhesive 61 may also be hidden between the front region 52 and the rear region 54.

The diapers 20 are contained in the package 100 so that the graphic 40 may face the window area 102. The window area 102 allows the graphic 40 to be at least partially visible. The reinforcement adhesive is covered by the ultraviolet barrier area 104. Because the ultraviolet barrier area 104 allows less ultraviolet transmittance than the window area 102, the reinforcement adhesive is protected from the ultraviolet ray. The external surface 44 of the diaper 20 is at least partially visible through the window area 102. When the graphic 40 faces the window area 102, the graphic 40 is also at least partially visible. A plurality of diapers 20 may be contained in the package 100 so that after one diaper 20 is taken out, another diaper 20 stacked adjacent to the diaper may be visible. When the graphic 40 of one diaper is different from that of another, the consumers may be pleased to retrieve the diapers 20 from the package 100.

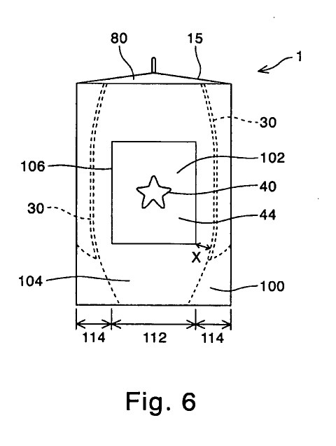

In the embodiment of FIG. 6, the leakage prevention adhesive 30 on the body facing surface of the backsheet 24 is covered by the ultraviolet barrier area 104 of the side panel 13. The size and the position of the window area 102 and the ultraviolet barrier area 104 may be determined so that the graphic 40 may be at least partially visible through the window area 102 and the leakage prevention adhesive 30 may be covered by the ultraviolet barrier area 104. In order to effectively allow the graphic 40 to be visible from outside, the window area 102 may be provided, for example relatively in the center region 112 of the side panel 13. In order to effectively protect the leakage prevention adhesive 30, the ultraviolet barrier area 104 may be provided to cover the side regions 114 of the side panel 13.

Referring to FIG. 6, the leakage prevention adhesive 30 is covered by an ultraviolet barrier area 104 for protection from the ultraviolet ray. A minimal distance X between the leakage prevention adhesive 30 and the window area 102 may be between about 5 mm and about 70 mm. Or the minimal distance X between the leakage prevention adhesive 30 and the window area 102 may be between about 10 mm and about 70 mm. The minimal distance X is determined by the length between one point of the leakage prevention adhesive 30 which is the closest to the window area 102 and the corresponding point of the border line 106.

The leakage prevention adhesive 30 may receive the ultraviolet ray which diffracts from the window area 102 even if the leakage prevention adhesive 30 does not face the window area 102 directly, when the minimal distance X is less than about 5 mm. When the minimal distance X is not less than about 5 mm, the leakage prevention adhesive 30 is protected by the ultraviolet ray without receiving the ultraviolet ray which diffracts from the window area 102. The diaper 20 and the package 100 are designed such that the leakage prevention adhesive 30 may be covered by the ultraviolet barrier area 104. When the minimal distance X is not less than about 5 mm, the diaper 20 may be contained in the package 100 in a way that the leakage prevention adhesive 30 is disposed to be covered by the ultraviolet barrier area enough even if the diaper 20 is contained off to the side.

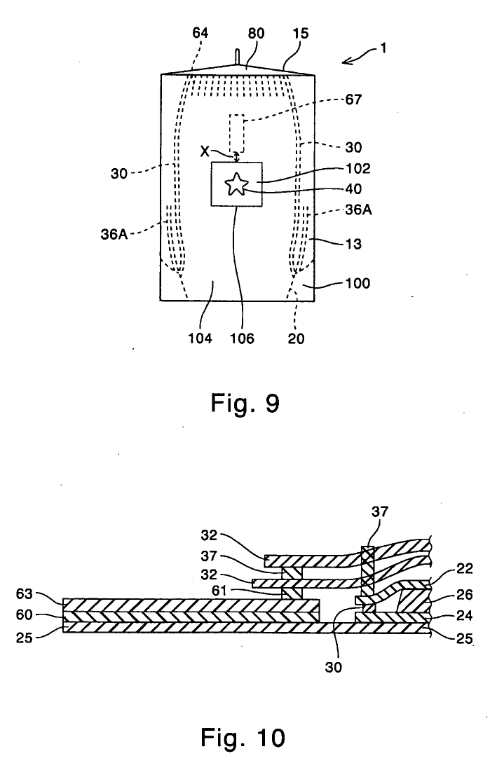

FIG. 9 illustrates an embodiment of a side view of the absorbent product 1 showing the relationship between the diaper 20 and the package 100. The diaper 20 is contained in the package 100 such that the disposable tape 67 is covered by the ultraviolet barrier area 104 of the side panel 13 and the disposable tape adhesive 70 is protected from the ultraviolet ray. In this embodiment, the window area 102 faces the rear region 54 of the diaper 20, and both the leakage prevention adhesive 30 and the disposable tape adhesive 70 are covered by the ultraviolet barrier area 104 and they are protected from the ultraviolet ray.

The diaper 20 may be contained in the package 100 with the side regions 58 being exposed out of the front region 52 and/or the rear region 54. In the embodiment, the ultraviolet barrier area 104 may be adjusted to cover the side region attachment adhesive 61. Both the leakage prevention adhesive 30 and the side region attachment adhesive 61 are protected by the ultraviolet barrier area 104. Alternatively, the diaper 20 may be contained in the package 100 with the side regions 58 being at least partially folded inside between the front region 52 and the rear region 54. Accordingly, the side region attachment adhesive 61 may be sandwiched between the front region 52 and the rear region 54. When the front region 52 and/or the rear region 54 protects the side region attachment adhesive 61 from the ultraviolet ray, the side region attachment adhesive 61 may partially face the window area 102 because the side region attachment adhesive 61 is protected by the front region 52 and/or rear region 54 from the ultraviolet ray.

The elastic adhesive 36 may be covered by the ultraviolet barrier area 104. Referring to FIG. 1, the elastic adhesive 36A is disposed transversely outside the leakage prevention adhesive 30. Therefore, if the leakage prevention adhesive 30 is located out of the window area 102, the elastic adhesive 36A is also disposed outside the window area 102 and the elastic adhesive 36A would be covered by the ultraviolet barrier area 104 and protected from the ultraviolet ray. Both the leakage prevention adhesive 30 and the elastic adhesive 36A are protected by the ultraviolet barrier area 104. Referring to FIG. 3, the elastic adhesive 36B is located transversely inside the leakage prevention adhesive 30. However, when the diaper 20 is viewed from outside, the elastic adhesive 36B is disposed inside the multilayer of the topsheet 22, the absorbent core 26, the backsheet 24 and the nonwoven 25. Therefore, the elastic adhesive 36B would be protected by the multilayer from the ultraviolet ray. Thus, even if the elastic adhesive 36B are located to face the window area 102, the elastic adhesive 36B would not be deteriorated when the diaper 20 is contained in the package 100.

As the window area 102 allows more light transmittance than the ultraviolet barrier area 104, the absorbent product 1 allows the graphic 40 to be visible from outside through the window area 102. As a result, the consumers can easily notice that the absorbent article 20 has a graphic 40 from outside the package 100 and the graphic 40 attracts the consumers. As the ultraviolet barrier area 104 allows less ultraviolet transmittance than the window area 102, the reinforcement adhesive having a basis weight of not less than about 10 g/m² is protected from the ultraviolet ray by the ultraviolet barrier area 104. The absorbent product 1 is useful for maintaining the quality of absorbent articles 20 (e.g. protection from discoloration, stiffening, loss of adhesibility and the like) because the reinforcement adhesive is not exposed to the ultraviolet ray even if the absorbent product 1 is displayed outside of the retailer shops.

### Test Methods

This section describes the methods for determining the light transmittance and the ultraviolet transmittance of a sheet material.

### 1. Light Transmittance

In the measurement, UV-VIS-NIR spectrophotometer (Shimadzu Corporation, UV-3600) is used to retrieve the desired light source of the experiment. This UV-VIS-NIR spectrophotometer includes a light source which has a halogen lamp, a sample holder, spectrometers, detector units which consist of a photomultiplier, InGaAs photo diode and PbS cell, and a computer. The light source is placed away from one side of the sample holder, while the detector is placed away from the other side of the sample holder in the path of the light.

In the measurement, the halogen lamp turns on. A sample sheet material is held by the sample holder so that it receives the light irradiated from the halogen lamp in the effective measurement area. The light passes through the sample sheet material and reaches the detector. The sample light volume (Vs) and the reference light volume (Vr) without a sample are then measured by the detector and recorded by the computer. This process is repeated for one sample sheet material at least three times and the average values of the light volumes (Vrav and Vsav) are calculated and recorded by the computer. The computer then calculates the light transmittance (LT) by the following formula:

LT(%)=(Vsav/Vrav)×100 (I)

### 2. Ultraviolet Transmittance Test

The measurement is carried out by the same procedure as the light transmittance test using UV-VIS-NIR spectrophotometer (Shimadzu Corporation, UV-3600) except that the ultraviolet ray in the wavelength range between 250 mm and 400 mm is retrieved. The ultraviolet transmittance is measured at the wavelength of 340 nm.

It is understood that the examples and embodiments described herein are for illustrative purpose only and that various modifications or changes will be suggested to one skilled in the art without departing from the scope of the present invention.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent product comprising:
(i) a package comprising a window area and an ultraviolet barrier area; the window area allowing more light transmittance than the ultraviolet barrier area and the ultraviolet barrier area allowing less ultraviolet transmittance than the window area, and
(ii) at least one disposable absorbent article contained in the package; the absorbent article extending in a longitudinal direction and in a transverse direction orthogonal to the longitudinal direction; the absorbent article comprising a liquid permeable topsheet, a liquid impermeable backsheet, an absorbent core disposed between the topsheet and the backsheet, and a reinforcement adhesive having a basis weight of not less than about 10 g/m²; the absorbent article comprising a graphic which is at least partially visible through the window area, and the reinforcement adhesive is covered by the ultraviolet barrier area.

2. The absorbent product of claim 1 wherein the absorbent core has longitudinal side edges and transverse end edges, and the reinforcement adhesive is a leakage prevention adhesive disposed transversely outside the longitudinal side edges of the absorbent core.

3. The absorbent product of claim 2 wherein the leakage prevention adhesive secures the backsheet to the topsheet.

4. The absorbent product of claim 2 wherein the disposable absorbent article further comprises a leg cuff, and the leakage prevention adhesive secures the backsheet to the leg cuff.

5. The absorbent product of claim 1 wherein the package comprises a front panel, a rear panel opposed to the front panel, a pair of side panels which connect the front panel and the rear panel, a top panel which connects the front panel, the rear panel and the side panels, and a bottom panel opposed to the top panel; the window area being formed on at least one of the side panels.

6. The absorbent product of claim 5 wherein the window area occupies between about 5% and about 80% of the total area of the side panel.

7. The absorbent product of claim 1 wherein the window area has a light transmittance of between about 15% and about 100%.

8. The absorbent product of claim 1 wherein the ultraviolet barrier area has a light transmittance of between about 0% and about 70%.

9. The absorbent product of claim 1 wherein the disposable absorbent article further comprises a disposable tape disposed on an external surface of the absorbent article and the disposable tape is covered by the ultraviolet barrier area.
